(19) United States
(12) Patent Application Publication
(10) Pub. No.: US 2024/0343821 A1
(43) Pub. Date: Oct. 17, 2024

(54) OFATUMUMAB FOR TREATING PEDIATRIC MS

(71) Applicant: NOVARTIS AG, Basel (CH)

(72) Inventors: Morten BAGGER, Basel (CH); Gordon GRAHAM, Basel (CH); Dieter Adrian HÄRING, Basel (CH); Martin MERSCHHEMKE, Basel (CH); Wendy SU, East Hanover, NJ (US)

(21) Appl. No.: 18/683,858

(22) PCT Filed: Jul. 27, 2022
(86) PCT No.: PCT/EP2022/071132

§ 371 Date: Feb. 15, 2024

Related U.S. Application Data
(60) Provisional application No. 63/254,684, filed on Oct. 12, 2021.
(60) Provisional application No. 63/233,524, filed on Aug. 16, 2021.

(30) Foreign Application Priority Data
Nov. 4, 2021 (EP) 21206466.1

(51) Int. Cl. C07K 16/28; A61K 39/00

(52) U.S. Cl. CPC C07K 16/2887; A61K 2039/505; A61K 2039/545; A61K 2039/55

(57) ABSTRACT

The invention concerns ofatumumab for use in the treatment or prevention of pediatric multiple sclerosis (MS). According to the invention, ofatumumab is administered during a loading dose regimen at weeks 0, 1, 2 of the dosage regimen; and ofatumumab is administered during a maintenance dose regimen starting at week eight of the dosage regimen and continuing thereafter every six weeks.

### FIELD OF THE INVENTION

The invention concerns ofatumumab for use in the treatment of pediatric multiple sclerosis (MS). According to the invention, ofatumumab is administered during a loading dose regimen at weeks 0, 1, 2 of the dosage regimen; and ofatumumab is administered during a maintenance dose regimen starting at week eight of the dosage regimen and continuing thereafter every six weeks.

### BACKGROUND OF THE INVENTION

The burden of pediatric MS to patients and their families is high. Pediatric MS patients experience re-occurring episodes of acute inflammatory activity and neurological abnormalities, which affect their quality of life severely. Pediatric onset of MS is associated with failure of age-expected brain growth during childhood/early adolescence and brain atrophy from midadolescence into adulthood. Eventually patients with pediatric onset of MS have a poor prognosis, become physically disabled and experience cognitive sequelae at a younger age compared to patients with adult onset MS. Unfortunately, current treatment avenues are very limited.

Thus, the unmet medical need in pediatric MS is high. To date, fingolimod (Gilenya®) is the only therapy (based on PARADIGMS), which demonstrated superior efficacy over interferon beta-la on disease activity. After PARADIGMS, TERIKIDS was conducted to investigate the efficacy and safety of teriflunomide in 166 pediatric patients. However, the study did not reach the statistical significance of the primary endpoint time to first clinical relapse. Therefore, fingolimod is the only approved therapy for pediatric MS in the USA. In the European Union (EU), interferon beta agents can be used in pediatric patients (with various age ranges) according to the drugs' approved labels. However, these EU-specific approvals were not based on prospective, randomized, controlled clinical studies such as PARADIGMS or TERIKIDS. As a result, the treatment options are not only limited, but also lack sufficient support and data from clinical studies.

In addition to the limited treatment options, such a lack of data is a problem when treatment is interrupted or changed. In this context, pediatric MS patients are particularly vulnerable and, thus, there is a need to reduce or minimize this vulnerability. Importantly, interruption or change of treatment is not a rare event in the treatment of pediatric patients. Reasons for treatment interruption or change include adverse effects, treatment failure, disease progression, disease regression, comorbidities, physiological and metabolic changes (such as menorrhea), and evolving patient preferences.

Accordingly, there is a need for medications that are effective as well as safe and tolerable for patients with pediatric MS.

Monoclonal antibodies (mAbs) directed against proteins expressed by B-cells, e.g. anti-CD20 antibodies, such as ofatumumab, ocrelizumab and rituximab, are high-efficacy disease-modifying therapies (DMTs) with a generally good safety profile (D'Amico et al 2019).

Ofatumumab (OMB157) is a human IgG1κ mAb, which targets CD20 expressed on B-cells and a subset of T-cells and unlike other anti-CD20 mAbs, ofatumumab binds to a distinct epitope on the CD20 molecule, a cell surface antigen present on pre-B and mature B lymphocytes, inducing potent B-cell lysis and depletion. Following cell surface binding to B lymphocytes, ofatumumab results in antibody-dependent cellular cytolysis and complement-mediated lysis.

Ofatumumab (Kesimpta®) is approved in the USA and Europe for the treatment of adults in relapsing forms of MS, to include clinically isolated syndrome, relapsing-remitting disease, and active secondary progressive disease.

### SUMMARY OF THE INVENTION

According to the invention it has unexpectedly been found that ofatumumab therapy is advantageous in pediatric MS patients as well as MS patients having a low body weight of at most 40 kg.

The present invention therefore provides ofatumumab for use in the treatment or prevention of multiple sclerosis in patients having at most 40 kg body weight and/or aged between 5 and 17 years.

The present invention further provides ofatumumab for use in the treatment or prevention of multiple sclerosis in patients having at most 40 kg body weight.

The present invention further provides ofatumumab for use in the treatment or prevention of multiple sclerosis in patients having at most 40 kg body weight and aged between 5 and 17 years.

### EMBODIMENTS

The present invention provides ofatumumab for use in the treatment or prevention, preferably in the treatment, of multiple sclerosis in patients having at most 40 kg body weight and/or aged between 5 and 17 years.

In a preferred embodiment the patients have a body weight of at most 40 kg. In another preferred embodiment the patients have a body weight of at least 25 kg, preferably the patients have a body weight of at least 25 kg and at most 40 kg.

In a preferred embodiment the patients are pediatric patients. In a preferred embodiment the patients are children. In another preferred embodiment, the patients are adolescents.

Preferably, a pediatric patient is between 5 and 17, more preferably from 10 to 17 years of age, i.e. from 10 to <18 years of age. In another embodiment a pediatric patient is from 10 to 12 years of age. In another embodiment a pediatric patient is between 5 and 14 years of age, preferably from 10 to 14 years of age. In another embodiment a pediatric patient is from 15 to 17 years of age, i.e. from >14 to <18 years of age.

In a preferred embodiment, a pediatric patient has a body weight of at most 40 kg. It is further preferred that a pediatric patient has a body weight of at least 25 kg, more preferably a pediatric patient has a body weight of at least 25 kg and at most 40 kg.

In a further preferred embodiment a pediatric patient is aged between 5 and 17, preferably 10 to <18 years of age, and has a body weight of at most 40 kg, preferably between 25 and 40 kg.

In a preferred embodiment of the present invention, ofatumumab is administered at a dose of 20 mg every 6 weeks, this dose is also referred to as maintenance dose.

Preferably, ofatumumab is administered parenterally, e.g. by epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural or intrasternal injection or infusion. The preferred route of administration is a subcutaneous injection (sc).

In a preferred embodiment of the present invention, ofatumumab is administered with a loading dose. The term loading dose is defined below. In a preferred embodiment, three loading doses are administered, preferably in week 0 and in week 1 and in week 2 after starting ofatumumab therapy. This means the first loading dose in week 0 constitutes the start of therapy. In an alternative preferred embodiment, three loading doses are administered on day 1, on day 5-9, preferably on day 7, and on day 12-16, preferably on day 14, after starting ofatumumab therapy. This means the first loading dose on day 1 constitutes the start of therapy.

According to the invention, ofatumumab is administered during a loading dose regimen, preferably comprising 20 mg ofatumumab, at weeks 0, 1, 2 of the dosage regimen; and ofatumumab is administered during a maintenance dose regimen, preferably comprising 20 mg ofatumumab, starting at week 8 of the dosage regimen and continuing thereafter every six weeks.

Thus, in a preferred embodiment ofatumumab is administered according to the following dosage regimen:
a) ofatumumab is administered as a loading dose, preferably comprising s.c. injections of 20 mg ofatumumab, at weeks 0, 1, 2 of the dosage regimen; and
b) ofatumumab is administered as a maintenance dose, preferably comprising s.c. injections of 20 mg ofatumumab, starting at week 8 of the dosage regimen and continuing thereafter every six weeks.

It has been completely surprising that this dosage regimen provides a safe and effective treatment in the patients defined in the claims, especially pediatric patients. This is because there are age-associated changes that are relevant for monoclonal antibodies (mAb) distribution and/or absorption, turnover and/or elimination.

Firstly, based on recent reports pointing out several processes that undergo age-associated changes and are relevant for mAb distribution it appears that the biodistribution of mAbs is affected by developmental changes. Thus, there is a well-known difference in the tissue water content of pediatric patients relative to that of adults. Thus, the fraction of total body volume available for distribution would be expected to be higher in pediatric patients for hydrophilic macromolecules such as monoclonal antibodies (mAbs). In addition, the perfusion rate of tissues in pediatric patients is usually higher than that of the corresponding tissues in adults. Furthermore, pediatric patients have a larger capillary surface area per unit tissue volume as well as a larger proportion of ‘leaky’ organs and tissues (e.g. liver, kidneys, and spleen) with an increased capillary permeability relative to their body size. Taken together, extravasation would be expected to be faster and concentration differences between vascular and extravascular spaces lower in pediatric patients compared to adults. Based on the increased extracellular fluid volume in pediatric patients compared to adults, as well as the higher perfusion rates that are assumed to be equally affected for plasma and lymph (˜0.2% of plasma flow rate), one would expect an increased absorption rate for mAbs in pediatric patients.

Secondly, the efficiency of recycling processes (such as the recycling process involving IgG receptor FcRn) as well as general age-associated differences in lysosomal protein turnover could be sources for differences in mAb elimination between pediatric patients and adults after correction for size differences. In this context, it has to be noted that protein turnover, i.e. catabolismin general, seems to be substantially higher in pediatric patients compared to adults. It would have been expected that the effects of pediatric age on these processes achieve clinically detectable differences in mAb elimination.

For these reasons, it would have been expected that age-related dose adjustments were necessary in the claimed patients, especially pediatric patients. The fact that the claimed dosage regimen is similar to the dosage regimen for adults is, therefore, completely unexpected.

Moreover, it has come as a surprise that the claimed dosage regimen can be safely and effectively administered subcutaneously (s.c.). This is because, for pediatric patients intravenous (IV) and to a lesser degree intramuscular (IM) administration is usually preferred.

Furthermore, it has been unexpected that there are no serious problems in terms of immunogenicity and endogenous anti-drug antibodies (ADA) in pediatric patients.

It has unexpectedly been found that the above-identified dosage regimen achieves a depletion of B cells below the threshold of 8 cells per microliter. This has been completely surprising because there are significant differences between adult patients and pediatric patients. These differences relate, inter alia, to physiology, metabolism, and body weight. In this context, it is also noted that there is a higher baseline B cell count in children compared to adults and, therefore, it would not have been expected that the claimed dosage regimen is able to consistently deplete B cells in patients as defined in the claims.

Most of the changes in B-cell subpopulations occur during childhood. There is a gradual decrease in the percentage and absolute numbers of naive B cells, beginning at the age of 18 months. On the other hand, there is an increase in memory B-cells. Switched memory B-cells, expressing CD27, have undergone somatic hypermutation and class-switch recombination in germinal centers. The percentage of CD27+ IgD− B cells increases until the age of 18 years and into adulthood. The population of CD19+ CD27+ IgD+ cells, often referred to as non-switched memory B cells, corresponds to circulating marginal zone B cells. These cells are involved in T-independent responses and play a crucial role in the control of infections due to encapsulated bacteria. There is a gradual increase in the percentage and absolute number of non-switched memory B cells between early childhood and adolescence. Without being bound by theory, it is believed that ofatumumab spares (a subset of) regulatory T or B cells, including marginal zone B cells. Therefore, it is plausible that the claimed dosage regimen for ofatumumab results in a safe and effective treatment in the patients defined in the claims.

On the other hand, the subject matter defined in the claims could not have been expected, based on the prior art. This is because age-related alterations such as those described above or those concerning maturation of regulatory T cells and other T-lymphocyte populations, would have given rise to the expectation that there are significant differences between pediatric patients and adults in terms of the maturation of immune system functionality and, therefore, in terms of processes involving immune cells such as immune reactivity. One exemplary process that might differ between pediatric patients and adults is ‘target-mediated drug disposition’ (TMDD), which represents an elimination pathway based on binding of mAbs to their target. Since the detection of these and other age-related differences and the assessment of their magnitude is complex and unpredictable, there was no expectation of success when using the claimed dosage regimen.

Generally, the present invention concerns the treatment of multiple sclerosis. In one embodiment of the invention, multiple sclerosis is relapsing-remitting multiple sclerosis (RRMS). In another embodiment of the invention, multiple sclerosis is primary progressive multiple sclerosis (PPMS). In a further embodiment of the invention, multiple sclerosis is secondary progressive multiple sclerosis (SPMS). In a further embodiment, multiple sclerosis is clinically isolated syndrome (CIS). RRMS is most preferred.

In another embodiment progressive forms of MS such as PPMS and SPMS are not included.

In the completed PARADIGMS study, fingolimod was compared to interferon (IFN) beta-1a (Avonex). Participants treated with IFN beta-1a experienced 120 MS relapses in 163 participant-years of exposure, while fingolimod treated participants experienced 25 MS relapses in 180 participant-years of exposure. The annualized relapse rate (ARR) (i.e. the number of MS relapses per year) was 0.122 in the fingolimod group and 0.675 in IFN beta-1a group. This corresponds to a reduction of 81.9% in ARR over IFN beta-0a (p<0.001) (Chitnis et al 2018).

In one embodiment of the invention ofatumumab is noninferior to interferons in terms of annualized relapse rates. especially in terms of maintaining the annualized relapse rates, preferably in terms of lowering the annualized relapse rates. In other words, the annualized relapse rate under ofatumumab treatment is at most the annualized relapse rate under interferons, i.e. under ofatumumab treatment the annualized relapse rate of the average patient is at most the annualized relapse rate of the average patient under interferons.

In a preferred embodiment ofatumumab is noninferior to interferon beta, particularly interferon beta selected from the group consisting of interferon-beta 1a, interferon-beta 1b, and pegylated forms thereof in terms of annualized relapse rates.

In particular, the annualized relapse rate (ARR) (i.e. the number of MS relapses per year) is less than 0.67, preferably less than 0.50, more preferably less than 0.25, even more preferably less than 0.15.

Preferably, ofatumumab leads to a reduction in ARR of at least 26% over IFN beta-1a (p<0.001), more preferably the reduction in ARR is at least 63%, more preferably at least 78%, in particular of at least 82%

In a preferred embodiment of the invention ofatumumab is noninferior to fingolimod in terms of annualized relapse rates, especially in terms of maintaining the annualized relapse rates, preferably in terms of lowering the annualized relapse rates. In other words, the annualized relapse rate under ofatumumab treatment is at most the annualized relapse rate under fingolimod, i.e. under ofatumumab treatment the annualized relapse rate of the average patient is at most the annualized relapse rate of the average patient under fingolimod.

Preferably, the ARR is at most 0.12. Preferably the ARR is less than 0.10, more preferably less than 0.08.

In another embodiment of the invention ofatumumab is noninferior to siponimod in terms of annualized relapse rates, in particular in terms of maintaining the annualized relapse rates, preferably in terms of lowering the annualized relapse rates. In other words, the annualized relapse rate under ofatumumab treatment is at most the annualized relapse rate under siponimod, i.e. under ofatumumab treatment the annualized relapse rate of the average patient is at most the annualized relapse rate of the average patient under siponimod.

In one embodiment of the invention ofatumumab is no worse (i.e. non-inferior) than fingolimod, in terms of ARR, using a non-inferiority margin of 2. Non-inferiority is assessed based on an estimated ARR-ratio (ofatumumab/fingolimod). In other words, the ARR-ratio of ofatumumab/fingolimod is lower than 2, preferably lower than 1.

In the PARADIGMS study the ARRratio (fingolimod/interferon) was 0.18 (95% CI: 0.11; 0.30) (Chitnis et al 2018). Taking the upper limit of the confidence interval as a conservative estimate, would result in ARR being 3.3-times higher on interferon versus fingolimod.

A margin of 2 versus fingolimod provides evidence of superiority of ofatumumab over interferons.

Optionally as an additional criterion, the primary objective will only be met if the posterior median of the ARR for ofatumumab is smaller than 0.3. This additional criterion protects against declaring non-inferiority in situations where the ARR would not be considerably lower than ARRs on interferons, which could otherwise occur if the ARR on comparator (fingolimod) would be higher than expected.

Still a further embodiment of the present invention is ofatumumab for use in the treatment or prevention of multiple sclerosis, wherein ofatumumab is noninferior to fingolimod in terms of the annualized T2 lesion rate, in other words wherein the annualized T2 lesion rate under ofatumumab is at most the annualized T2 lesion rate under fingolimod, preferably wherein the annualized T2 lesion rate under ofatumumab is reduced as compared to fingolimod.

Still a further embodiment of the present invention is ofatumumab for use in the treatment or prevention of multiple sclerosis, wherein ofatumumab is noninferior to fingolimod in terms of neurofilament light chain (NfL) concentrations in serum. In other words, under ofatumumab treatment neuroaxonal damage as measured by the NfL concentrations in serum is at most as severe as under fingolimod treatment, preferably under ofatumumab treatment neuroaxonal damage as measured by the NfL concentrations in serum is reduced as compared to fingolimod treatment.

In a preferred embodiment the invention relates to ofatumumab for use in the treatment or prevention of multiple sclerosis, wherein neuroaxonal damage as measured by the NfL concentrations in serum is reduced as compared to fingolimod.

Another embodiment of the invention is ofatumumab for use in the treatment or prevention of multiple sclerosis, wherein ofatumumab is noninferior to fingolimod in terms of safety and tolerability as determined by at least one of the following criteria:
frequency and severity of treatment emergent adverse events (TEAEs),
Columbia Suicide Severity Rating Scale (C-SSRS),
12-lead ECG,
laboratory and ophthalmological data,
pulmonary function tests,
vital signs.

Another embodiment of the invention relates to ofatumumab for use in the treatment or prevention of multiple sclerosis, wherein ofatumumab is noninferior to fingolimod in terms of immunogenicity and endogenous anti-drug antibodies (ADA).

In a preferred embodiment of the present invention, ofatumumab for use in treating MS is used in a long-term treatment. The term long-term treatment indicates that ofatumumab is used over an extended period of time. For example, ofatumumab can be used for more than 2 years, 3 years, 4 years, 5, years, 10 years. Ofatumumab might be used up to 5 years, 10 years, 15 years, 20 years or for life.

In a preferred embodiment of the present invention, ofatumumab is administered at a dose of 20 mg every 6 weeks in patients having at most 40 kg body weight or in patients having at most 40 kg body weight and aged between 5 and 17 years. When the patients reach a body weight of more than 40 kg or when the patients attain the age of 18 and reach a body weight of more than 40 kg the dosage regimen is simply switched to a dose of 20 mg every 4 weeks.

This allows to continue MS treatment despite changing circumstances such as age or body weight gain by only a slight modification of the treatment protocol without the need to switch the disease-modifying drug. This is particularly advantageous since switching the disease-modifying drug may require a wash out period and/or can be associated with the occurrence of adverse events.

In a preferred embodiment of the present invention, a premedication is administered to the patient before the first dose of ofatumumab is administered. Preferably, the premedication comprises a compound selected from acetaminophen, antihistamines and steroids. Methylprednisolone may be a preferred steroid. 100 mg iv may be a preferred dose. Preferably, the premedication is administered 30 to 60 minutes prior to an ofatumumab injection.

In a particularly preferred embodiment, no premedication is administered prior to the first dose of ofatumumab.

In a preferred embodiment of the present invention, ofatumumab is administered as the sole active ingredient for treating MS. In other words, ofatumumab is preferably the only disease-modifying drug that is administered.

Generally, side effects and adverse events associated with B cell-depleting therapies such as ocrelizumab therapy are reported to be associated with a reduction of immunoglobulins (e.g. IgG). In the present invention it was surprisingly found that ofatumumab therapy is advantageous compared to other B cell-depleting therapies because it does not cause reduction of immunoglobulins (e.g. IgG) in the long run and thus opens up new avenues for patients under long-term treatment.

Hence, in a preferred embodiment of the present invention ofatumumab is used in the treatment of MS, wherein ofatumumab is administered to patients with known risk factors for malignancies. In another preferred embodiment of the present invention ofatumumab is used in the treatment of MS, wherein ofatumumab is administered to patients who are being actively monitored for recurrence of malignancy. In an alternative embodiment of the present invention ofatumumab is used in the treatment of MS, wherein ofatumumab is administered to patients with a known active malignancy.

The MSIS-29 (see definition below) is a clinically useful and scientifically sound measure of the impact of MS from the patient's perspective suitable for clinical studies and epidemiological studies. It is considered a reliable, valid and responsive PRO (Patient Reported Outcomes) measure that complements other indicators of disease severity used to improve our understanding of the impact of MS.

In the present invention, it was unexpectedly found that administration of ofatumumab leads to an advantageous reduction of the MS impact scale MSIS-29 as defined below.

In this regard a further subject of the present invention is ofatumumab for use in the treatment or prevention of relapsing multiple sclerosis, wherein ofatumumab reduces the MSIS-29 score. Preferably, ofatumumab reduces the MSIS-29 score by at least 1.5, more preferably at least 2.0, still more preferably at least 2.5 within 24 months. The reduction might be up to 3.0 or 3.5 or 4.0.

In one embodiment of the invention, an ofatumumab composition is formulated in accordance with routine procedures as a pharmaceutical composition adapted for intravenous administration to human beings. Typically, compositions for intravenous administration are solutions in sterile isotonic aqueous buffer. Where suitable, the composition may also include a solubilizing agent and a local anesthetic, such as lignocaine, to ease pain at the injection site. Generally, the ingredients are supplied either separately or mixed together in a unit dosage form, for example as a dry lyophilized powder or water-free concentrate, in a hermetically sealed container, such as an ampoule or sachet, indicating the quantity of active agent.

Where the composition is to be administered by infusion, in particular by subcutaneous injection (s.c.), it can be dispensed with an infusion bottle containing sterile pharmaceutical grade water or saline.

Where the composition is administered by injection, an ampoule of sterile water for injection or saline can be provided so that the ingredients may be mixed prior to administration.

In one embodiment, a formulation for ofatumumab can be formulated according to a formulation disclosed in WO 2009/009407.

In one embodiment, ofatumumab is formulated in an antibody formulation wherein ofatumumab is present in an amount of about 20-300 mg/mL, 50-300 mg/mL, 100-300 mg/mL, 150-300 mg/mL, 200-300 mg/mL or 250-300 mg/mL, preferably at 50 mg/ml.

In one embodiment, ofatumumab is formulated in an antibody formulation wherein the formulation comprises 10 to 100 mM sodium acetate, 25 to 100 mM sodium chloride, 0.5 to 5% arginine free base, 0.02 to 0.2 mM EDTA, 0.01 to 0.2% polysorbate 80 and adjusted to pH 5.0 to 7.0. Preferably, the ofatumumab formulation comprises 50 mM sodium acetate, 51 mM sodium chloride, 1% arginine free base, 0.05 mM EDTA, 0.02% polysorbate 80 and adjusted to pH 5.5.

The preferred dosage regimen of ofatumumab is:
initial dosing or loading dose of 20 mg by subcutaneous injection at Weeks 0, 1 and 2, followed by
subsequent dosing or maintenance dose of 20 mg by subcutaneous injection starting at week eight of the dosage regimen and continuing thereafter every six weeks.

If an injection of ofatumumab is missed, it should preferably be administered as soon as possible without waiting until the next scheduled dose. Subsequent doses should be administered at the recommended intervals.

In one embodiment, the ofatumumab formulation is provided in a pre-filled syringe or in an auto-injector, preferably a single-dose pre-filled syringe or a single-dose pre-filled auto-injector. Preferably, a pre-filled auto-injector designed for s.c. administration is used.

In a preferred embodiment, ofatumumab injection is a sterile, preservative-free solution for subcutaneous use. Preferably, each 20 mg/0.4 mL prefilled pen or prefilled syringe delivers 0.4 mL of solution. Preferably, each 0.4 mL contains 20 mg of ofatumumab and arginine (4 mg), disodium edetate (0.007 mg), polysorbate 80 (0.08 mg), sodium acetate trihydrate (2.722 mg), sodium chloride (1.192 mg) and Water for Injection, USP with a pH of 5.5. Hydrochloric acid may be added to adjust pH.

In a preferred embodiment the ofatumumab formulation is intended for patient self-administration, preferably by subcutaneous injection.

In a preferred embodiment said formulation is administered in the abdomen, thigh or outer upper arm subcutaneously. In a preferred embodiment said formulation is not administered into moles, scars or areas where the skin is tender, bruised, red, hard or not intact.

In an embodiment, the first injection of said ofatumumab formulation may be performed under the guidance of a healthcare professional. If injection-related reactions occur, symptomatic treatment is recommended. Before administration, the pen or prefilled syringe is preferably removed from the refrigerator and allowed to reach room temperature, e.g. for about 15 to 30 minutes. In a preferred embodiment, the ofatumumab formulation of the present invention is a clear to slightly opalescent and colorless to slightly brownish-yellow solution available as follows:
Injection: 20 mg/0.4 mL in a single-dose prefilled pen, e.g. Sensoready® pen
Injection: 20 mg/0.4 mL in a single-dose prefilled syringe.

In a preferred embodiment of the invention, ofatumumab administration is delayed in patients with an active infection, e.g. COVID-19, until the infection is resolved. Alternatively, the ofatumumab can be administered during an infection, e.g. during a COVID-19 infection. Thus, ofatumumab administration can be continued during an infection, e.g. during a COVID-19 infection.

In another preferred embodiment of the invention, the level of immunoglobulins at the beginning, during and after discontinuation of treatment with ofatumumab are monitored as clinically indicated until B-cell repletion. Discontinuing ofatumumab treatment is considered if a patient develops a serious opportunistic infection or recurrent infections if immunoglobulin levels indicate immune compromise.

In one embodiment of the invention, ofatumumab is used in a patient who has been treated with a disease-modifying therapy other than ofatumumab, wherein the drug of the earlier disease-modifying therapy is selected from ocrelizumab, rituximab, fingolimod, teriflunomide, interferon beta, and glatiramer acetate.

A further subject of the present invention is ofatumumab for use in the treatment of pediatric multiple sclerosis, wherein the treatment is a long-term treatment and wherein the serum IgG level is maintained within a range, wherein said range is essentially the same in untreated patients.

In the context of the present invention, “untreated patients” refers to pediatric patients diagnosed with MS or clinically isolated syndrome (CIS) and which are not administered a B cell and/or T cell inhibitor. In a preferred embodiment the untreated patients present IgG levels in a range from 600 to 2500 mg/dl.

Another subject of the present invention is ofatumumab for use in the treatment of pediatric multiple sclerosis, wherein pediatric patients having a lowered serum IgG level are treated.

Still another subject of the present invention is ofatumumab for use in the treatment of multiple sclerosis, wherein pediatric patients having risk factors associated with serum Ig levels, in particular serum IgG levels, are treated.

In an embodiment of the invention, ofatumumab is not administered to pediatric patients having an active HBV infection, particularly having an active HBV infection confirmed by positive results for Hepatitis B surface antigen [HBsAg] and anti-HBV tests. Ofatumumab may or may not be administered to pediatric patients who are negative for HBsAg and positive for Hepatitis B core antibody [HBcAb+] or are carriers of HBV [HBsAg+].

In another embodiment of the invention ofatumumab is not administered to pediatric patients having an acute or chronic hepatitis A, B, C and/or E infection.

In another embodiment of the invention ofatumumab is not administered to pediatric patients having received any live or live-attenuated vaccines (including for varicella zoster virus or measles) within 4 weeks prior to the intended first administration of ofatumumab.

A further subject of the invention is a method for treating multiple sclerosis, said treatment comprising administering ofatumumab to a patient in need thereof, wherein the patient
i) has at most 40 kg body weight and/or
ii) is aged between 5 and 17 years.

A further subject of the present invention is a method for the manufacture of a medicament for use in the treatments described above.

### Definitions

The terms “treatment” or “treat” can be defined as the application or administration of e.g. ofatumumab to a patient, where the purpose is to abolish, reduce or alleviate the symptoms of a disease such as multiple sclerosis (MS). In particular, the term “treatment” comprises the achievement of a clinically meaningful effect for the patient, for example the achievement of a clinically meaningful reduction of the annual relapse rate when treating RMS. The term further includes preventing moving to a progressive form of MS.

The term “patient” preferably refers to a human patient, e.g. a patient having a disorder or at risk of having a disorder described herein. Preferably, the patient is a pediatric patient. According to the invention the treatment described herein is suitable for an individual patient as well as a patient population.

The term “pediatric patient” covers patients until the age of 18 and, hence, includes children as well as adolescents. Preferably, a pediatric patient is between 5 and 17, more preferably from 10 to <18 years of age (i.e. they have not yet had their 18^{th} birthday). More preferably, a pediatric patient has a body weight of at most 40 kg. It is further preferred that a pediatric patient has a body weight of at least 25 kg, more preferably a pediatric patient has a body weight of at least 25 kg and at most 40 kg.

More preferably, a pediatric patient is aged between 5 and 17 and has a body weight of at most 40 kg, preferably between 25 and 40 kg.

The term “children” as used herein refers to individuals aged between 5 and 12 years of age and the term “adolescents” refers to to individuals aged between 13 and <18 years of age.

The term “adverse event” (AE) can relate to any untoward medical occurrence in a patient or clinical investigation wherein the subject is administered a pharmaceutical product which does not necessarily have a causal relationship with this treatment. An adverse event (AE) can therefore be any unfavourable and unintended sign (including an abnormal laboratory finding), symptom or disease temporally associated with the use of a medicinal (investigational) product, whether or not related to the medicinal (investigational) product.

### RRMS

Relapsing-remitting multiple sclerosis (RRMS) may be characterized by relapses, defined as a new neurologic deficit or episode of neurologic worsening lasting longer than 24 h, preferably in the absence of fever or infection.

There may be no apparent progression of the disease during the periods of remission. At different points in time, RRMS might be further characterized as either active (with relapses and/or evidence of new MRI activity) or not active, as well as worsening (a confirmed increase in disability over a specified period of time following a relapse) or not worsening. Reference is made to Lublin, Neurology. 2014 Jul. 15; 83 (3): 278-286.

### RMS

The term RMS (relapsing multiple sclerosis) encompasses RRMS, SPMS and clinically isolated syndrome (CIS).

### Primary Progressive MS (PPMS)

PPMS can be characterized by worsening neurologic functions (accumulation of disability) from the onset of symptoms, without early relapses or remissions. PPMS can be further characterized at different points in time as either active (with an occasional relapse and/or evidence of new MRI activity) or not active, as well as with progression (evidence of disease worsening on an objective measure of change over time, with or without relapse or new MRI activity) or without progression. References is made to Lublin 2014.

Each person's experience with PPMS will be unique. PPMS can have brief periods when the disease is stable, with or without a relapse or new MRI activity, as well as periods when increasing disability occurs with or without new relapses or lesions on MRI.

### Secondary Progressive MS (SPMS)

SPMS follows an initial relapsing-remitting course. Most people who are diagnosed with RRMS will eventually transition to a secondary progressive course in which there is a progressive worsening of neurologic function (accumulation of disability) over time. SPMS can be further characterized at different points in time as either active (with relapses and/or evidence of new MRI activity) or not active, as well as with progression (evidence of disease worsening on an objective measure of change over time, with or without relapses) or without progression. Reference is made to Lublin 2014.

Each person's experience with SPMS will be unique. SPMS follows after relapsing-remitting MS. Disability gradually increases over time, with or without evidence of disease activity (relapses or changes on MRI). In SPMS, occasional relapses may occur, as well as periods of stability.

Siponimod, sold under the brand name Mayzent®, is a selective sphingosine-1-phosphate receptor modulator for oral use that is used for multiple sclerosis (MS).

### Relapse

Relapses can be defined as a new neurologic deficit or episode of neurologic worsening, preferably lasting longer than 24 h. In other words, relapses can be regarded as discrete episodes (in the art also referred to as “attacks,” “flare-ups” or “exacerbations”) of neurologic dysfunction, preferably lasting at least 24 h. Usually, relapses are followed by full or partial recovery and a period in which there is no symptom progression or accumulation of disability (remission).

The term “annualized relapse rate” (ARR) relates to the number of MS relapses per year, in particular to the number of MS relapses of the average patient per year. The term “average patient” as used herein relates to the average behavior of a treated patient population.

B cell as used herein may relate to a type of white blood cell of the lymphocyte subtype. B cells function in the humoral immunity component of the adaptive immune system by secreting antibodies such as immunoglobulins (e.g. IgG). Additionally, B cells may present antigens and secrete cytokines. B cells, unlike the T cells and natural killer cells, express B cell receptors (BCRs) on their cell membrane. BCRs allow the B cell to bind to a specific antigen against which it will initiate an antibody response.

T cell as used herein may relate to a type of lymphocyte which develops in the thymus gland. T cells can be distinguished from other lymphocytes by the presence of a T cell receptor on the cell surface.

### Clinically Isolated Syndrome (CIS):

Clinically isolated syndrome (CIS) may refer to a single clinical attack of central nervous system (CNS) inflammatory demyelinating symptoms that are suggestive of multiple sclerosis (MS). CIS presentations can be monofocal or multifocal and typically may involve the optic nerve, brainstem, cerebellum, spinal cord or cerebral hemispheres. Reference is made to Miller et al, Clinically isolated syndromes, Lancet Neurol. 2012; 11:157-169.

### Gd+ Lesion

Gadolinium (“contrast”) is a chemical compound that is injected into a person's vein during an MRI scan. Gadolinium normally cannot pass from the bloodstream into the brain or spinal cord due to the blood-brain barrier. But during active inflammation within the brain or spinal cord, as during an MS relapse, the blood-brain barrier is disrupted, allowing gadolinium to pass through. Gadolinium can then enter the brain or spinal cord and leak into an MS lesion, lighting it up and creating a highlighted spot on an MRI. Such an MS lesion is called gadolinium-enhanced lesion or Gd+ lesion.

### T1 and T2 Lesions

T1 and T2 relate to different MRI methods used to generate magnetic resonance images. Specifically, T1 and T2 refers to the time taken between magnetic pulses and recording of an image. These different methods are used to detect different structures or chemicals in the central nervous system. T1 and T2 lesions refer to whether the lesions were detected using either the T1 or T2 method. A T1 MRI image supplies information about current disease activity by highlighting areas of active inflammation. A T2 MRI image provides information about disease burden or lesion load (the total amount of lesion area, both old and new).

The term “annualized T2 lesion rate” relates to the number of new or newly enlarging T2 lesions on MRI per year.

### EDSS

The Expanded Disability Status Scale (EDSS) is a method of quantifying disability in multiple sclerosis and monitoring changes in the level of disability over time.

The EDSS scale ranges from 0 to 10 in 0.5-unit increments that represent higher levels of disability. Scoring is based on an examination by a neurologist.

EDSS steps 1.0 to 4.5 refer to people with MS who are able to walk without any aid and is based on measures of impairment in eight functional systems (FS):
pyramidal—muscle weakness or difficulty moving limbs
cerebellar—ataxia, loss of balance, coordination or tremor
brainstem—problems with speech, swallowing and nystagmus
sensory—numbness or loss of sensations
bowel and bladder function
visual function—problems with sight
cerebral functions—problems with thinking and memory
other.

A functional system (FS) represents a network of neurons in the brain with responsibility for particular tasks. Each FS is scored on a scale of 0 (no disability) to 5 or 6 (more severe disability). Reference is made to Kurtzke J F. Rating Neurologic Impairment in Multiple Sclerosis: An Expanded Disability Status Sclale (EDSS). Neurology. 1983 November; 33 (11): 1444-52.

### Multiple Sclerosis Impact Scale (MSIS-29)

The MSIS-29 version 2 is a 29-item, self-administered questionnaire that includes 2 domains: physical and psychological. Responses were captured on a 4-point ordinal scale ranging from 1 (not at all) to 4 (extremely), with higher scores reflecting greater impact on day-to-day life. The MSIS-29 takes about 5 minutes to complete and the questions are designed to determine the patient's views about the impact of MS on their day-to-day life during the past 2 weeks. Reference is made to Hobart J and Cano S (2009), “Improving the evaluation of therapeutic interventions in multiple sclerosis: the role of new psychometric methods”, Health Technol Assess; 13(12):iii, ix-x, 1-177. NS RO to Hobart J, Lamping D, Fitzpatrick R, et al (2001), “The Multiple Sclerosis Impact Scale (MSIS-29): a new patient-based outcome measure”, Brain; 124(Pt 5):962-73.

### Ofatumumab:

Ofatumumab is a human monoclonal antibody for the CD20 protein. Ofatumumab may bind specifically to both the small and large extracellular loops of the CD20 molecule. The Fab domain of ofatumumab may bind to the CD20 molecule and the Fc domain mediates immune effector functions to result in B-cell lysis in vitro. In particular, ofatumumab is a recombinant human monoclonal immunoglobulin G1 (IgG1) antibody that binds to human CD20 expressed on e.g. B cells. Ofatumumab is produced in a murine NS0 cell line and consists of two IgG1 heavy chains and two kappa light chains with a molecular weight of approximately 146 kDa.

Ofatumumab is described in EP 1 558 648 B1 and EP 3 284 753 B1. Further reference is made to the description in the drugbank.ca, accession number DB06650 and to WHO Drug Information, Vol. 20, No. 1, 2006. In an embodiment, the protein chemical formula is C₆₄₈₀H₁₀₀₂₂N₁₇₄₂O₂₀₂₀S₄₄ and the protein average weight is about 146100 Da. In the US ofatumumab is marketed under the tradename Kesimpta®.

The metabolic pathway of ofatumumab can be degradation to small peptides and amino acids by ubiquitous proteolytic enzymes. Ofatumumab might be eliminated in two ways: a target-independent route as with other IgG molecules and a target-mediated route that is related to binding to B cells.

The half-life of ofatumumab at steady state can be approximately 16 days, in particular following subcutaneous administration of repeated 20 mg doses.

Ofatumumab preferably does not share a common clearance pathway with chemical drugs that are metabolized by the cytochrome P450 system or other drug metabolizing enzymes. Preferably, ofatumumab is not involved in the regulation of the expression of drug metabolizing enzymes.

### Loading Dose

A loading dose is an initial dose of a drug, preferably an initial higher dose that may be given at the beginning of a treatment (e.g. a DMT) before transitioning to a maintenance dose, preferably being lower or administered at greater intervals than the loading dose.

### Disease-Modifying Therapy (DMT)

The term “disease-modifying therapy” is used because there is still no curative treatment for multiple sclerosis (MS), but several disease-modifying drugs (DMDs) have been approved for MS. Generally, DMTs for RMS decrease the frequency and/or seriousness of relapses. Thus, DMTs are not a cure for RMS patients, but they can reduce how many relapses someone has and how serious they are. DMTs include, without limitation treatment with DMDs such as interferon beta, glatiramer acetate, teriflunomide, mitoxantrone, dimethyl fumarate, cladribine, fingolimod, siponimod, ponesimod, alemtuzumab, daclizumab, natalizumab, ofatumumab, ocrelizumab and rituximab.

### EXAMPLE

### Phase III Clinical Study Comparing the Efficacy and Safety of Ofatumumab and Siponimod Versus Fingolimod in Pediatric Patients with Multiple Sclerosis

The purpose and rationale of the study is to demonstrate the efficacy and to assess safety/tolerability of ofatumumab and siponimod in pediatric multiple sclerosis (MS) patients (aged 10-<18 years) versus fingolimod.

### Objective

The primary objective is to demonstrate the non-inferiority of ofatumumab and/or siponimod as compared to fingolimod as assessed by annualized relapse rate (ARR) in the target pediatric MS participants treated for up to 2-years.

Secondary objectives particularly include to demonstrate the superiority of ofatumumab and/or siponimod as compared to historical interferon ß-la data, assessed by annualized relapse rate (ARR of confirmed relapses).

Secondary objectives further include:
to evaluate the effects of ofatumumab and/or siponimod versus fingolimod on the number of new or newly enlarging T2 lesions, assessed by the number of new or newly enlarging T2 lesions on MRI per year (annualized T2 lesion rate)
to evaluate the effects of ofatumumab and/or siponimod versus fingolimod on neurofilament light chain (NfL) concentrations, assessed by Neurofilament light chain (NfL) concentration in serum
to evaluate the pharmacokinetic (PK) properties of ofatumumab and siponimod (and its metabolite M17) in pediatric MS patients, assessed by ofatumumab and siponimod and (metabolite M17) plasma concentrations
to evaluate immunogenicity (ofatumumab), assessed by the proportion of participants with anti-ofatumumab antibodies
to evaluate the safety and tolerability of ofatumumab and siponimod, assessed by adverse events, Columbia Suicide Severity Rating Scale (C-SSRS), ECG, laboratory and ophthalmological data, pulmonary function tests and vital signs

### Study Design

The study randomizes approximately 180 participants in a 1:1:1 randomization allocation ratio (60 participants randomized to s.c. ofatumumab, 60 to oral siponimod and 60 to oral fingolimod). This includes the targeted enrollment of at least 5 participants with a body weight (BW) of ≤40 kg and at least 5 participants between the ages of 10 and 12 years in each of the ofatumumab and siponimod arm.

The study is composed of three parts:
a Core Part, which includes the screening period and the double-blind treatment period
an Extension Part, which includes a transitioning period with double-blind treatment
followed by an open label treatment period
a Post-treatment follow-up part.

### Analysis

Efficacy assessments will include the following assessments:
MS relapse
EDSS
MRI
Neurofilament light chain (NfL) concentration in serum
Symbol Digit Modalities Test (SDMT)
B-cell

The primary endpoint of the study is the annualized relapse rates (ARR) which is defined as the average number of confirmed relapses per year (i.e. the total number of confirmed relapses divided by the total days in the study multiplied by 365.25).

Endpoints for secondary objectives include the annualized relapse rate (ARR of confirmed relapses), the number of new or newly enlarging T2 lesions on MRI per year (annualized T2 lesion rate), Neurofilament light chain (NfL) concentration in serum, ofatumumab and siponimod and (metabolite M17) plasma concentrations, the proportion of participants with anti-ofatumumab antibodies, adverse events, Columbia Suicide Severity Rating Scale (C-SSRS), ECG, laboratory and ophthalmological data, pulmonary function tests and vital signs.

ARR is analyzed using a Bayesian primary analysis model with only confirmed relapses.

The analysis of secondary endpoints include efficacy and/or pharmacodynamic endpoints such as comparison against historical interferon data, annualized rate of new/newly enlarging T2 lesions.

### Ofatumumab and Siponimod PK/Pd Relationships

An exploratory PK/PD analysis of absolute B cell counts is performed if deemed relevant after assessment of the main results of the study.

### No Evidence of Disease Activity

Proportion of participants free of clinical and MRI disease activity (no evidence of disease activity; NEDA-3) is analyzed cross-sectionally at year 1 and year 2 in a logistic regression model adjusting for treatment, T2 lesion volume and age at baseline. NEDA-3 is defined as no 3mCDW, no confirmed MS relapse and no new or enlarging T2 lesions on any MRI scan compared to baseline. The analysis considers only those participants who were followed-up to the assessment time point in the analysis (e.g. only participants with ≥12 months of follow-up in the 12-month assessment of disease freedom, etc.). Intermediate missing values (e.g. due to missing MRI assessments) are considered not free of disease activity.

### 3-Month Confirmed Disability Worsening

A 3-month confirmed disability worsening (3mCDW) is defined as an increase from baseline in EDSS sustained for at least 3 months. This means that after a scheduled or unscheduled visit at which the patient fulfills the disability worsening criterion, all EDSS assessments (scheduled or unscheduled) need to also fulfill the worsening criteria until the worsening (“the event”) can be confirmed at the first scheduled visit that occurs 3-months after the onset of the worsening, or later. Censoring occurs in all participants who did not experience a 3mCDW event in the study (censoring also occurs in participants who had a “tentative” disability worsening that could not be confirmed due to an early discontinuation or any another reason). The censoring time is defined as the time from the first dose to the last available EDSS assessment.

Further assessed are subject reported outcomes including:
Child Health Utility 9D (CHU9-D)
Pediatric Quality of Life Inventory (PedsQL)
PedsQL Multidimensional Fatigue Scale

The invention is further characterized by the following embodiments:
1. Ofatumumab for use in the treatment or prevention of multiple sclerosis in patients
   i) having at most 40 kg body weight and/or
   ii) aged between 5 and 17 years.
2. Ofatumumab for use in the treatment or prevention of multiple sclerosis in patients having at most 40 kg body weight.
3. Ofatumumab for use in the treatment or prevention of multiple sclerosis in patients
   i) having at most 40 kg body weight and
   ii) aged between 5 and 17 years.
4. Ofatumumab for use according to any one of the preceding embodiments, wherein the patients are pediatric patients.
5. Ofatumumab for use according to any one of the preceding embodiments, wherein the patients are aged between 10 and 17 years.
6. Ofatumumab for use according to any one of the preceding embodiments, wherein ofatumumab is administered according the following dosage regimen:
   a) ofatumumab is administered as a loading dose at weeks 0, 1, 2 of the dosage regimen; and
   b) ofatumumab is administered as a maintenance dose starting at week eight of the dosage regimen and continuing thereafter every six weeks.
7. Ofatumumab for use according to embodiment 6 wherein the loading dose and the maintenance dose comprise s.c. injections of 20 mg ofatumumab.
8. Ofatumumab for use according to any one of the preceding embodiments, wherein the treatment or prevention achieves a depletion of B cells below the threshold of 8 cells per microliter.
9. Ofatumumab for use according to any one of the preceding embodiments, wherein multiple sclerosis is relapsing-remitting multiple sclerosis (RRMS).
10. Ofatumumab for use according to any one of the preceding embodiments, wherein multiple sclerosis is clinically isolated syndrome (CIS).
11. Ofatumumab for use according to any one of the preceding embodiments, wherein ofatumumab is noninferior to fingolimod in terms of annualized relapse rates.
12. Ofatumumab for use according to any one of the preceding embodiments, wherein ofatumumab is noninferior to fingolimod in terms of maintaining the annualized relapse rates, preferably in terms of lowering the annualized relapse rates
13. Ofatumumab for use according to any one of the preceding embodiments, wherein ofatumumab is noninferior to interferons in terms of annualized relapse rates.
14. Ofatumumab for use according to any one of the preceding embodiments, wherein ofatumumab is noninferior to interferons in terms of maintaining the annualized relapse rates, preferably in terms of lowering the annualized relapse rates.
15. Ofatumumab for use according to embodiment 13 or 14, wherein interferon is interferon beta, particularly interferon beta selected from the group consisting of interferon-beta 1a, interferon-beta 1b, and pegylated forms thereof.
16. Ofatumumab for use according to any one of the preceding embodiments, wherein ofatumumab is noninferior to siponimod in terms of annualized relapse rates.
17. Ofatumumab for use according to any one of the preceding embodiments, wherein ofatumumab is noninferior to siponimod in terms of maintaining the annualized relapse rates, preferably in terms of lowering the annualized relapse rates.
18. Ofatumumab for use according to any one of the preceding embodiments, wherein the annualized relapse rate of the average patient is less than 0.67.
19. Ofatumumab for use according to any one of the preceding embodiments, wherein the annualized relapse rate of the average patient is at most 0.12.
20. Ofatumumab for use according to any one of the preceding embodiments, wherein the ARR is reduced by at least 26% as compared to interferons, more preferably the reduction in ARR is at least 63%, more preferably at least 78%, in particular at least 82%.
21. Ofatumumab for use according to any one of the preceding embodiments, wherein ofatumumab is used in a patient who has been treated with a disease-modifying therapy other than ofatumumab, wherein the drug of the earlier disease-modifying therapy is selected from ocrelizumab, rituximab, fingolimod, teriflunomide, interferon beta, and glatiramer acetate.
22. Ofatumumab for use according to any one of the preceding embodiments, wherein the treatment is a long-term treatment.
23. Ofatumumab for use according to any one of the preceding embodiments, wherein a premedication is administered to the patient before the first dose of ofatumumab is administered.
24. Ofatumumab for use according to any one of the preceding embodiments, wherein the premedication comprises acetaminophen, antihistamines and/or steroids.
25. Ofatumumab for use according to any one of the preceding embodiments, wherein the premedication is administered 30 to 60 minutes prior to an ofatumumab injection.
26. Ofatumumab for use according to any one of the preceding embodiments, wherein no premedication is administered prior to the first dose of ofatumumab.
27. Ofatumumab for use according to any one of the preceding embodiments, wherein a patient acutely or previously infected by COVID-19 is treated.
28. Ofatumumab for use according to any one of the preceding embodiments, wherein the treatment is continued during COVID-19 infection.
29. Ofatumumab for use according to any one of the preceding embodiments, wherein the treatment is interrupted during COVID-19 infection und continued after the infection is resolved.
30. Ofatumumab for use according to any one of the preceding embodiments, wherein ofatumumab is noninferior to fingolimod in terms of the annualized T2 lesion rate.
31. Ofatumumab for use according to any one of the preceding embodiments, wherein the annualized T2 lesion rate is reduced as compared to fingolimod.
32. Ofatumumab for use according to any one of the preceding embodiments, wherein ofatumumab is noninferior to fingolimod in terms of neurofilament light chain (NfL) concentrations in serum.
33. Ofatumumab for use according to any one of the preceding embodiments, wherein neuroaxonal damage as measured by the NfL concentrations in serum is reduced as compared to fingolimod.
34. Ofatumumab for use according to any one of the preceding embodiments, wherein ofatumumab is noninferior to fingolimod in terms of safety and tolerability as determined by at least one of the following criteria:
   frequency and severity of treatment emergent adverse events (TEAEs),
   Columbia Suicide Severity Rating Scale (C-SSRS),
   12-lead ECG,
   laboratory and ophthalmological data,
   pulmonary function tests,
   vital signs.
35. Ofatumumab for use according to any one of the preceding embodiments, wherein the mean number of Gd-enhancing T1 lesions per year is reduced as compared to fingolimod.
36. Ofatumumab for use according to any one of the preceding embodiments, wherein the risk of 3-month confirmed disability worsening (3mCDW) is reduced as compared to fingolimod.
37. Ofatumumab for use according to any one of the preceding embodiments, wherein the risk of 6-month confirmed disability worsening (6mCDW) is reduced as compared to fingolimod.
38. Ofatumumab for use according to any one of the preceding embodiments, wherein ofatumumab is noninferior to fingolimod in terms of immunogenicity and endogenous anti-drug antibodies (ADA)
39. A method for treating multiple sclerosis, said treatment comprising administering ofatumumab to a patient in need thereof, wherein the patient
   i) has at most 40 kg body weight and/or
   ii) is aged between 5 and 17 years.
40. A method for the manufacture of a medicament for use in the treatment of multiple sclerosis, wherein the patients to be treated
   i) have at most 40 kg body weight and/or
   ii) are aged between 5 and 17 years.

1. Ofatumumab for use in the treatment or prevention of multiple sclerosis in patients
i) having at most 40 kg body weight and/or
ii) aged between 5 and 17 years.

2. Ofatumumab for use in the treatment or prevention of multiple sclerosis in patients having at most 40 kg body weight.

3. Ofatumumab for use in the treatment or prevention of multiple sclerosis in patients
i) having at most 40 kg body weight and
ii) aged between 5 and 17 years.

4. Ofatumumab for use according to any one of the preceding claims, wherein the patients are pediatric patients.

5. Ofatumumab for use according to any one of the preceding claims, wherein the patients are aged between 10 and 17 years.

6. Ofatumumab for use according to any one of the preceding claims, wherein ofatumumab is administered according the following dosage regimen:
c) ofatumumab is administered as a loading dose at weeks 0, 1, 2 of the dosage regimen; and
d) ofatumumab is administered as a maintenance dose starting at week eight of the dosage regimen and continuing thereafter every six weeks.

7. Ofatumumab for use according to claim 6 wherein the loading dose and the maintenance dose comprise s.c. injections of 20 mg ofatumumab.

8. Ofatumumab for use according to any one of the preceding claims, wherein ofatumumab is noninferior to fingolimod, siponimod or interferon beta in terms of maintaining the annualized relapse rates, preferably in terms of lowering the annualized relapse rates.

9. Ofatumumab for use according to any one of the preceding claims, wherein the annualized relapse rate of the average patient is less than 0.67, preferably at most 0.12.

10. Ofatumumab for use according to any one of the preceding claims, wherein the ARR is reduced by at least 26% as compared to interferons, more preferably the reduction in ARR is at least 63%, more preferably at least 78%, in particular at least 82%.

11. Ofatumumab for use according to any one of the preceding claims, wherein ofatumumab is used in a patient who has been treated with a disease-modifying therapy other than ofatumumab, wherein the drug of the earlier disease-modifying therapy is selected from ocrelizumab, rituximab, fingolimod, teriflunomide, interferon beta, and glatiramer acetate.

12. Ofatumumab for use according to any one of the preceding claims, wherein the treatment is a long-term treatment.

13. Ofatumumab for use according to any one of the preceding claims, wherein a patient acutely or previously infected by COVID-19 is treated.

14. Ofatumumab for use according to any one of the preceding claims, wherein the treatment is continued during COVID-19 infection.

15. Ofatumumab for use according to any one of the preceding claims, wherein ofatumumab is noninferior to fingolimod in terms of the annualized T1 lesion rate, in terms of T2 lesion rate, in terms of neurofilament light chain (NfL) concentrations in serum, in terms of immunogenicity and endogenous anti-drug antibodies (ADA) and/or in terms of safety and tolerability as determined by at least one of the following criteria:
frequency and severity of treatment emergent adverse events (TEAEs),
Columbia Suicide Severity Rating Scale (C-SSRS),
12-lead ECG,
laboratory and ophthalmological data,
pulmonary function tests,
vital signs.
